(19) United States
(12) Patent Application Publication
(10) Pub. No.: US 2012/0220887 A1
(43) Pub. Date: Aug. 30, 2012

(54) CPR MONITORING SYSTEM WITH ACTIVE BACKBOARD

(76) Inventor: Helge Fossan, Stavanger (NO)

(21) Appl. No.: 13/344,650

(22) Filed: Jan. 6, 2012

(30) Foreign Application Priority Data
Jan. 13, 2011 (NO) 20110055

(51) Int. Cl. A61B 5/113

(52) U.S. Cl. 600/534; 73/866.4

(57) ABSTRACT

The invention relates to a monitoring system for monitoring CPR performance and a reference unit for use in the system. The system comprises a measuring unit for positioning on the chest of a patient or manikin monitoring the movements applied to the chest, wherein the system also comprises a reference unit, the reference unit comprising sensors for measuring the movements thereof, wherein said units comprises means for wireless communication, one of the units comprising a signal analyzing means and a signal receiver coupled thereto for receiving said wireless communication, and the other unit comprising a signal transmitter for transmitting the measured signals to the signal receiver.

### BACKGROUND OF THE INVENTION

1. Technical Field

This invention relates to a CPR (cardio-pulmonary resuscitation) monitoring system comprising a first unit for positioning on the chest of a patient or manikin monitoring the movements applied to the chest.

2. History of Related Art

Measuring of CPR movements at the chest of a user is well known and described e.g. in WO2006/006871, U.S. Pat. No. 7,476,206 and US2008/312565. As is mentioned in WO2006/006871 and U.S. Pat. No. 7,476,206 a reference measurement may be performed at the back of the patient in order to compensate for movements of the patient as a whole. Thus a measurement of the movement of the chest relative to the back is obtained. The reference measurements are either performed using measuring units placed in the bed or in a so called backboard positioned beneath the patient, both being connected to a separate system calculating the relative movements. Thus the system is dependent on a fairly complex system located in hospitals or other places having the necessary equipment.

### SUMMARY OF THE INVENTION

It is an object of the present invention to provide a monitoring system being compact and thus possible to use in situ where the patient is placed, and to provide relevant measurements of the CPR performance. This is obtained with a system as mentioned above and being characterized as discussed in the accompanying claims.

According to the preferred embodiment of the invention the processing means, receiver and other circuitry such as storage and coupling means to eternal equipment is placed in the measuring unit. This way the invention provides a solution where reference unit is a low cost unit which can be used mounted on a backboard or independently, and which may be disposable.

### BRIEF DESCRIPTION OF THE DRAWINGS

The invention will be discussed below with reference to the accompanying drawings, illustrating the invention by way of examples.

FIG. 1 illustrates the preferred embodiment of the system according to the invention.

FIG. 2 illustrates a first embodiment of the reference unit according to the invention.

FIG. 3 illustrates a second embodiment of the reference unit according to the invention.

FIG. 4 illustrates the circuitry of the reference unit according to the invention.

FIG. 5a,b illustrates alternative embodiments of the backboard and reference unit according to the invention.

### DETAILED DESCRIPTION OF ILLUSTRATIVE EMBODIMENTS OF THE INVENTION

Embodiment(s) of the invention will now be described more fully with reference to the accompanying Drawings. The invention may, however, be embodied in many different forms and should not be construed as limited to the embodiment(s) set forth herein. The invention should only be considered limited by the claims as they now exist and the equivalents thereof.

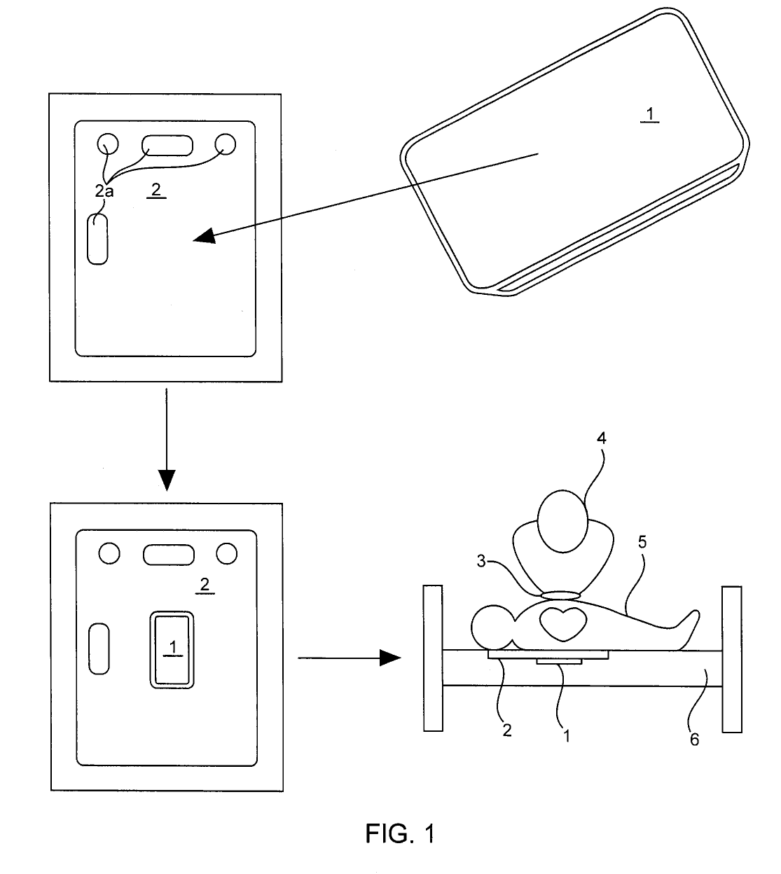

FIG. 1 gives a general overview of the use of the preferred embodiment of the invention, where a reference unit 1 is attached to an existing back board 2, which is placed under the patient 5. The illustrated backboard has grips etc 2a for convenience and possibly for indicating the orientation of the backboard.

The CPR is performed by a person 4 using a measuring unit 3, e.g. as described in WO2006/006871 or US2008/312565, being adapted to received the measured signals from the reference unit 1. Being positioned under the patient 5 the movements measured at the reference unit is the movements of the patient relative to the bed 6, thus providing a correction factor that may be used to remove the influence of these movements from the movements measured at the measuring unit 3.

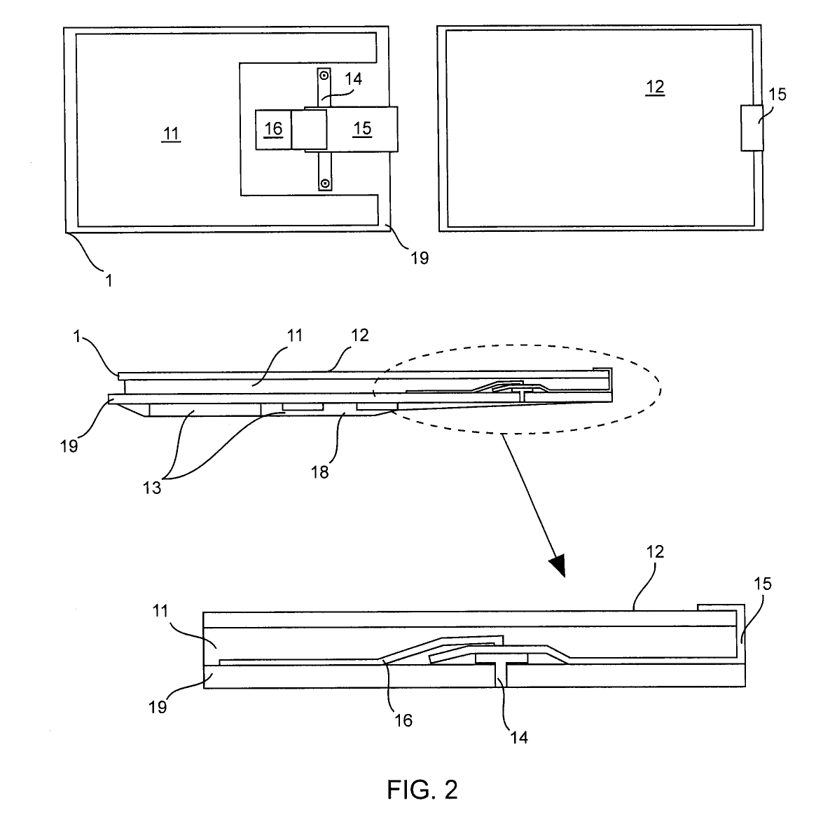

The illustration in FIG. 2 shows a possible inexpensive design of a disposable Back Board module 1. The drawing shows the module as seen from above without and with a protective liner 12, and in a cross section. Basically the module 1 is just a PCB 19 with adhesive foam 11 on the back and a simple activation switch 14, 15, 16. The activation switch will power the module when the protective film 15 is removed just prior to application on the Back Board and consists in this embodiment of a spring metal 16 providing electrical contact with a copper track 14 when pressurized. The protective film may be removed at the same time or after the removal of the protective liner 12 covering the protective foam 11 being fastened to the back board 2.

The module 1 also includes electronic circuitry, including a movement sensor such as an acceleration sensor, and battery 13 driving the circuitry, all preferably covered by a protective coating material 18 for protection.

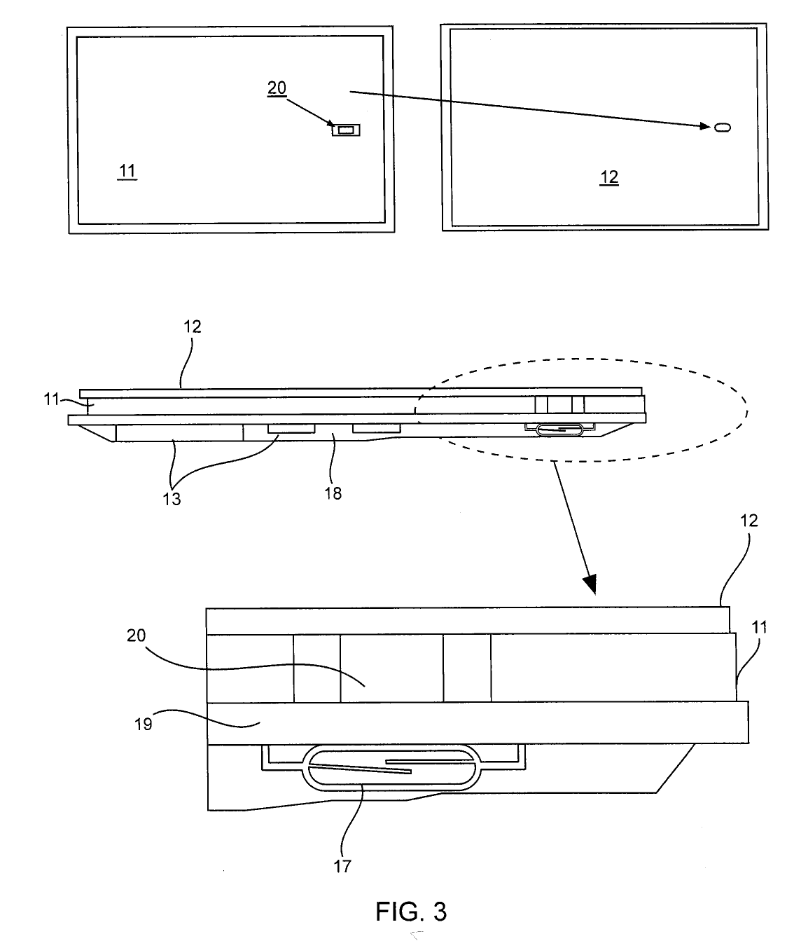

The illustration in FIG. 3 shows an alternative version of a possible design of a disposable Back Board module 1. As above the module is also just a PCB with adhesive foam 11 on the back and a simple activation switch 17, 20. The activation switch is a reed switch 17 which will be activated by the removal of a magnet 20 attached to the liner 12.

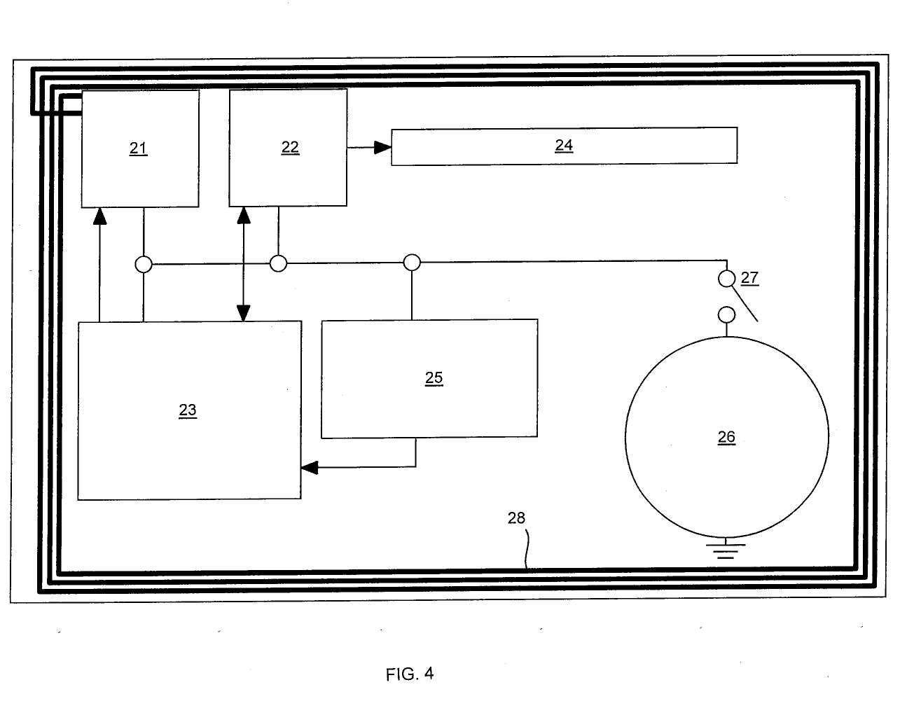

The illustration in FIG. 4 shows a possible circuit topology of a module 1, comprising, a battery 26 or similar power sources, an accelerometer 25 for sensing the movements of the unit coupled to a micro controller 23 which may include calculation means, system clock and calculation means depending on the chosen communication form, e.g. continuous or package based communication. The switch 27 may correspond to the switches 14, 16 or 17, 20 in FIGS. 2 and 3.

Both RFID 21, 28 and Bluetooth 22, 24 units are shown. The RFID unit includes processing means 21 and an antenna 28 surrounding the circuitry and the Bluetooth unit comprises processing means and a shorter antenna 24. The module could have both RFID and Bluetooth which would provide flexibility, but this may have an undesirable price penalty. One possibility would be to produced the units with circuit layout for both, but only assemble one of the two.

Bluetooth is a standard communication system which can provide “real time” communication and transfer of acceleration samples but at the cost of relatively high power consumption. With respect to the new so-called Low power Bluetooth standard which may be preferably in this use this is more uncertain since this standard is based upon “burst” transmission to save power.

RFID is another standard that is not a real time sample transfer media. With RFID the data to be transferred must be handled by “data packs”. With respect to data transfer from the module this means that the recoded depth from CPR can be transmitted as information packages. The sampled information may then be pretreated so as to comprise information packages with predetermined information such as compression number and corresponding compression depth or as average depth for the last chosen number of compressions.

The information processing may depend on the use and also of the chosen cost of the reference unit 1. A continuous communication of the information from the accelerometer to the measuring unit, e.g. by Bluetooth, might be contemplated, but at the cost of power supply. Preferably the information is transmitted in packages using the RFID or Low Power Bluetooth protocols.

In the preferred embodiment of the invention the information from the accelerometer is received by calculation means in the micro controller 23 which may calculate the mean movement over a chosen time to generate a correction factor to be sent to the measuring unit. Usually this is only necessary once as the movements relative to the bed do not change if the compression movements applied on the patient is the same. This way the communication time and thus power consumption is reduced as the signal may only be transmitted after the first chosen number of registered compressions.

The microcontroller may also be adapted to monitor the measured movements over time and generate a new correction factor if there is a change in the movements, and it may provide a measure of any movements, e.g. if position in an ambulance, when mounted on the backboard before the compressions start, for example after being activated by the switch 14, 15, 16 (FIG. 2) or 17, 20 (FIG. 3).

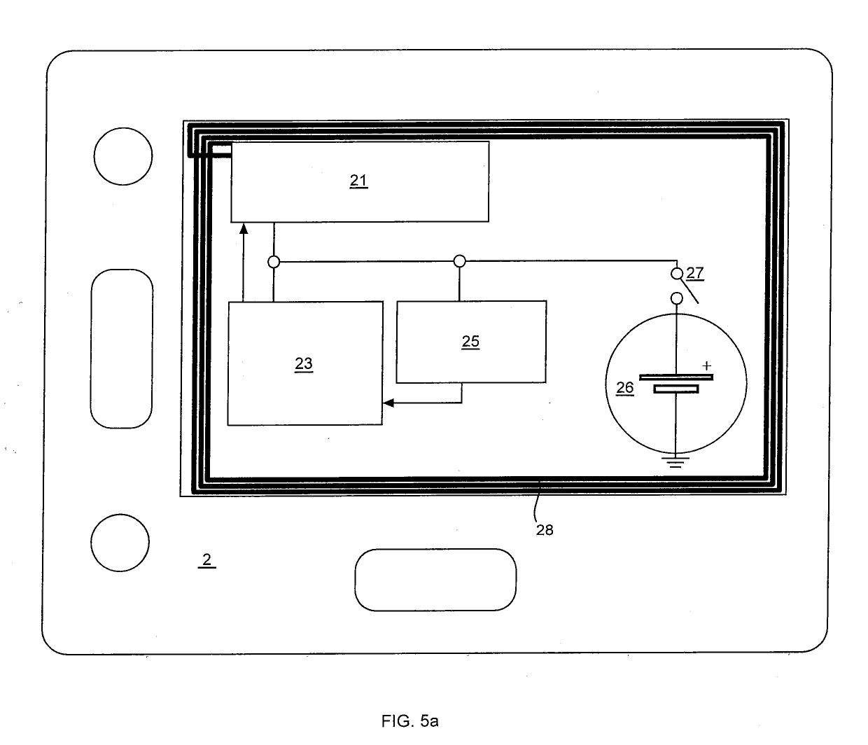

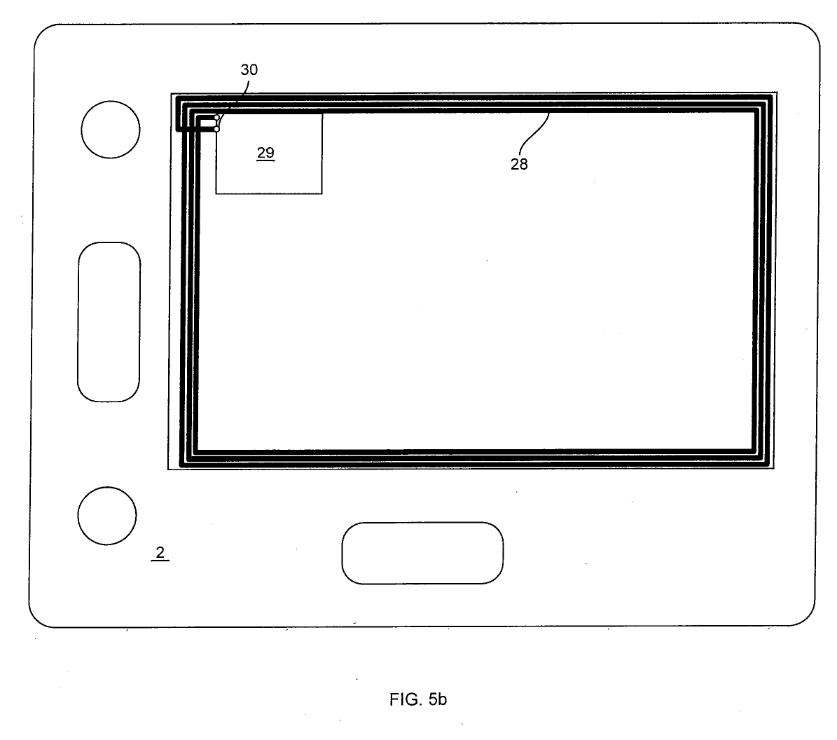

FIG. 5a, 5b illustrates two versions on the invention where the reference unit 1 is adapted to provide two way communication with the measuring unit 3. This may for example enable requests from the measuring unit for new reference signals, or reactivating the reference unit, or adjusting signal strength, equalizing the signal or adjusting the sample time window.

In order to provide sufficient reach for the transmitted signals the RFID antenna 28 is enlarged so as to cover a circumference being comparable to the size of the back board 2. In FIG. 5a the complete reference unit is included, while in FIG. 5b the antenna 28 is included into the back board having contact points 30 for coupling to corresponding coupling point in the reference unit 29. While these solutions may require special backboards the advantage two-way communication between the reference unit and the backboard may justify the additional cost.

Thus to summarize the present invention preferably relates to a monitoring system for monitoring CPR performance. The system comprises a measuring unit for positioning on the chest of a patient or manikin monitoring the movements applied to the chest, and also comprises a reference unit being positioned at a different place not on the patient, but preferably on the opposite side of the patient relative to the measuring unit. If the measuring unit is positioned on the chest the reference unit is preferably positioned at the back of the patient. The reference unit comprising sensors for measuring its movements in the direction of the CPR compressions.

Both the measuring unit and reference unit comprises means for wireless communication, one of the units comprising a signal analyzing means and a signal receiver coupled thereto for receiving said wireless communication, and the other unit comprising a signal transmitter for transmitting the measured signals to the signal receiver.

Preferably the wherein the analyzing means is positioned in the measuring unit receiving signals transmitted from the reference unit. In order to reduce power consumption the reference unit preferably comprises calculation means being adapted to calculate chosen characteristics of the measured movements, for example mean movement depth and compressions per time unit, and communicate the calculated information to the analyzing means. This way the information is compressed thus limiting the power transmission from the reference unit and thus also the power necessary for driving the reference unit.

The analyzing means is adapted to calculate the relative movement between the units fro the measured movements at the measuring unit and the reference unit, so as to provide an indication of the quality of the compressions, which may either be stored in the measuring unit for later use, communicated to the person performing the compressions or transmitted immediately to external instruments for further analysis.

The signal transmitter and receiver are radio transmitter and receiver, preferably using RFID signals if the micro controller in the reference unit has calculation means for providing information packages to be sent. It is also possibly to have both transmitters and receivers in both units for providing two-way communication between them, e.g. to send control signals to the reference unit for ordering a new correction factor.

The reference unit is adapted to be mounted on a backboard being positioned under the patient or manikin during CPR, e.g. fastened to the backboard using an adhesive.

The movements sensors in said reference unit includes at least one accelerometer for measuring the movements of the reference unit in one direction, i.e. in the direction of the performed compressions, but may be able to measure movements in two or three directions. It may comprise a switch for activating the reference unit, e.g. when being mounted on the back board.

The measuring unit may also comprise a wireless power transmitter and the reference unit comprises a receiver and a rechargeable power source, the reference unit also including circuitry charging the power source from the received signals from the measuring unit, e.g. using RFID signals as a power source. Alternatively the reference unit may be changed in a special charging unit, either through electrical coupling or RF signals when not in use.

Preferably the reference unit according to the invention is adapted to be used in a CPR monitoring system comprising a measuring unit to be position on the chest of a patient or manikin during chest compressions. The reference unit comprising mounting means for mounting on a plane object, e.g. a back board, to be positioned under said patient or manikin, a movement sensor for measuring movements in the direction perpendicular to the plane object, the reference unit also comprising communication means for communicating a signal corresponding to the measured movement to the measuring unit. The communication means may either be an antenna included in the reference unit or coupling means for connecting to a corresponding antenna positioned on a backboard.

As mentioned above the reference unit may comprise calculation means for calculating the mean movement depth over a chosen time fro the measured movements, the communication means being adapted to transmit said mean movement depth to said measuring unit.

The reference unit of the preferred embodiment also comprises an adhesive foam for adhering to the plane object, the foam being provided with a protective foil being adapted to activate a switch activating the reference unit when removed.

Although various embodiments of the method and apparatus of the present invention have been illustrated in the accompanying Drawings and described in the foregoing Detailed Description, it will be understood that the invention is not limited to the embodiments disclosed, but is capable of numerous rearrangements, modifications and substitutions without departing from the spirit of the invention as set forth herein.

1. A monitoring system for monitoring CPR performance comprising a measuring unit for positioning on the chest of a patient or manikin monitoring the movements applied to the chest, wherein the system also comprises a reference unit, the reference unit comprising sensors for measuring the movements thereof, wherein said units comprises means for wireless communication, one of the units comprising a signal analyzing means and a signal receiver coupled thereto for receiving said wireless communication, and the other unit comprising a signal transmitter for transmitting the measured signals to the signal receiver.

2. The monitoring system according to claim 1, wherein the analyzing means is positioned in the measuring unit.

3. The monitoring system according to claim 1, wherein the calculation means are positioned in the reference unit being adapted to calculate chosen characteristics of the measured movements, e.g. mean movement depth and compressions per time unit, and communicate the calculated information to the analyzing means.

4. The monitoring system according to claim 1, wherein the signal processing is adapted to calculate the relative movement between the units.

5. The monitoring system according to claim 1, wherein the signal transmitter and receiver are radio transmitter and receiver, e.g. using RFID signals.

6. The monitoring system according to claim 1, wherein both units comprise transmitter and receiver, so as to provide two-way communication between the two.

7. The monitoring system according to claim 1, wherein the reference unit is adapted to be mounted on a backboard being positioned under the patient or manikin during CPR.

8. The monitoring system according to claim 7, wherein the reference unit is adapted to be mounted on a backboard using an adhesive.

9. The monitoring system according to claim 1, wherein the sensors for measuring the movements in said reference unit include an accelerometer for measuring the movements of the reference unit.

10. The monitoring system according to claim 1, wherein the reference unit comprises a switch for activating the reference unit.

11. The monitoring system according to claim 1, wherein the measuring unit comprises a wireless power transmitter and the reference unit comprises a receiver and a rechargeable power source, the reference unit also including circuitry charging the power source from the received signals from the measuring unit.

12. A reference unit for use in a CPR monitoring system comprising a measuring unit to be position on the chest of a patient or manikin during chest compressions, comprising mounting means for mounting on a plane object, to be positioned under said patient or manikin, a movement sensor for measuring movements in the direction perpendicular to the plane object, the reference unit also comprising communication means for communicating a signal corresponding to the measured movement to the measuring unit.

13. The reference unit according to claim 12, comprising calculation means for calculating the mean movement depth over a chosen time from the measured movements, the communication means being adapted to transmit said mean movement depth to said measuring unit.

14. The reference unit according to claim 12, comprising an adhesive foam for adhering to the plane object, the foam being provided with a protective foil being adapted to activate a switch activating the reference unit when removed.

15. The reference unit according to claim 12, wherein the plane object is a back board.
